(19) United States
(12) Patent Application Publication
(10) Pub. No.: US 2026/0355319 A1
(43) Pub. Date: Nov. 12, 2026

(54) DICARBOXYLIC ACID CONCENTRATES

(71) Applicant: Vantage Specialty Ingredients, Inc., Warren, NJ (US)

(72) Inventors: James Vincent Gruber, Washington, NJ (US); Xiang Chen, Somerset, NJ (US); Phatcharada Phondee, Morristown, NJ (US); Yurah Kim, Somerset, NJ (US)

(73) Assignee: Vantage Specialty Ingredients, Inc., Warren, NJ (US)

(21) Appl. No.: 19/572,184

(22) Filed: Mar. 19, 2026

Related U.S. Application Data
(63) Continuation of application No. 19/479,247, filed on Apr. 16, 2024.
(60) Provisional application No. 63/462,390, filed on Apr. 27, 2023.

(51) Int. Cl. A61K 47/12; A61K 8/362; A61K 8/42; A61K 31/192; A61K 47/18; A61Q 19/00; A61Q 19/10

(52) U.S. Cl. CPC A61K 47/12; A61K 8/362; A61K 8/42; A61K 31/192; A61K 47/18; A61Q 19/007; A61Q 19/10; A61K 2800/10

(57) ABSTRACT

A substantially-anhydrous, dermatologic concentrate consisting essentially of (a) a long-chain alkyl dicarboxylic acid having poor water solubility and having six to twelve carbon atoms selected from the group consisting of adipic acid, azelaic acid, sebacic acid, and dodecanedioc acid; and (b) an amidoamine. The substantially-anhydrous, dermatologic concentrate can include salicylic acid.

### BACKGROUND OF THE INVENTION

Azelaic Acid is a dicarboxylic acid found in whole grain cereals-wheat, rye, barley- and is produced by Malassezia furfur, a commensal yeast fungus that is part of the human skin microbiome.

Azelaic acid is reported in the scientific literature to exhibit antibacterial, keratolytic, comedolytic, and anti-oxidant activity. It inhibits microbial cellular protein synthesis and reduces the thickness of the stratum corneum.

For treatment of acne vulgaris and inflammatory papules and pustules of mild to moderate rosacea, two prescription strengths are available in different dosage forms-20% active cream (AZELEX®) and 15% active gel or foam (FINACEA®). Lower “non-prescription” strength—10% active—preparations are marketed for cosmetic indications including as a brightener that reduces dullness, uneven tone, and textural irregularities.

Other reported dermatologic benefits from use of azelaic acid include reducing the production of sebum in the sebaceous gland, reducing hair loss or thinning, including by acting as a competitive inhibitor of the reduction of testosterone to dihydrotestosterone.

Two representative, non-prescription, skincare products that contain azelaic acid are: THE ORDINARY® 10% Azelaic Acid Suspension and PAULA'S CHOICE® 10% Azelaic Acid Booster (also contains 0.5% salicylic acid).

U.S. Pat. No. 6,734,210, expired, describes the difficulties of solubilizing azelaic acid. Water “only marginally dissolves” azelaic acid—to a maximum of 0.24% by weight (w/w)—a concentration that is “not likely” to be clinically effective. Isopropanol is described as “good” solvent but “unsatisfactory” because it can be drying. Ethanol renders azelaic acid unstable at “normal temperatures” resulting in a “totally ineffective composition”.

U.S. Pat. Nos. 4,713,394 and 4,885,282, similarly characterize the instability of azelaic acid when dissolved in ethyl alcohol.

In July 2020, Azeco Cosmeceuticals published a “Technical Information File” that presents guidance on formulating with azelaic acid and a generalized solution to the limited solubility of azelaic acid in aqueous systems-namely, incorporating azelaic acid in a final formulation using a “premix”. According to Azeco, the most “straightforward” “premix” is created by dissolving azelaic acid in ethanol or isopropanol. However, Azeco notes that for many dermatologic preparations, these alcohols are not preferred; and, accordingly, diols are used
namely propanediol (available, for example, under the tradename Zemea® from DuPont Tate & Lyle Bio Products Company, LLC, Wilimington, Delaware), 1,3-butylene glycol, and pentylene glycol (available, for example, under the tradename Pentiol Green+ from Minasolve SAS, Beuvry-La-Forêt, France). The Azeco formulation guidance also comments that “glycerin and glycerin/water mixtures are suitable solvent systems; [but] the maximum concentration [s] [of] water in this solvent mixture are [sic] not properly defined and not reported in the literature.”

There has been and remains a need for concentrates of azelaic acid (and other long-chain alkyl dicarboxylic acids having six to twelve carbon atoms that have poor solubility in water) that can be incorporated into topical formulations having a hydrophilic carrier (water, or combination of water and water-miscible ingredients (e.g., hydroglycolic or hydroalcoholic mixture) or mixture of alcohols and/or glycols) without forming a visible precipitate.

There has been and remains a need for concentrates that include both azelaic acid (or another long-chain alkyl dicarboxylic acids having six to twelve carbon atoms that is poorly soluble in water) and salicylic acid. Those needs are met by the substantially-anhydrous, dermatologic concentrates of the present invention disclosure.

### SUMMARY OF THE INVENTION

A first aspect of the present invention disclosure is directed to substantially-anhydrous, dermatologic concentrates that consist essentially of (or consist of)
(a) a long-chain alkyl dicarboxylic acid having poor solubility in water and having six to twelve carbon atoms selected from the group consisting of adipic acid, azelaic acid, sebacic acid, and dodecanedioc acid, preferably azelaic acid; and
(b) an amidoamine, preferably an alkylamido alkylamine, more preferably a fatty acid amidopropyl dimethylamine, most preferably cocamidopropyl dimethylamine.

A second aspect of the present invention disclosure is directed to substantially-anhydrous, dermatologic concentrates that consist essentially of, and consist of, substantially-anhydrous, dermatologic concentrates in accordance with the first aspect of the present invention disclosure that further consist essentially of (or consist of) salicylic acid.

When a substantially-anhydrous, dermatologic concentrate in accordance with the first or second aspects of the present invention disclosure is added to a hydrophilic carrier (e.g., water) there is no visible precipitate of either the long-chain alkyl dicarboxylic acid and/or salicylic acid.

### BRIEF DESCRIPTION OF THE DRAWINGS

FIGS. 1 and 2 presents comparative results of Enzyme-Linked Immunosorbent Assay (ELISA) for Tumor Necrosis Factor-alpha (TNF-α) after application of substantially-anhydrous, dermatologic concentrates of the present invention.

FIG. 3 presents comparative results of ELISA for Caspase-1 (CASP-1) after application of substantially-anhydrous, dermatologic concentrates of the present invention.

### DETAILED DESCRIPTION OF INVENTION

As used in the present invention disclosure, “poor water solubility” means<30 g/L water at 25 °C. See, Fiume, M M et al, “Final Report of the Cosmetic Ingredient Review Expert Panel on the Safety Assessment of Dicarboxylic Acids, Salts, and Esters” International Journal of Toxicology 31 (Supplement 1) 5S-76S (2012) (DOI: 10.1177/1091581812447203).

In describing concentrates of the present invention disclosure, “substantially anhydrous” is to be understood to mean other than water of hydration contained in the constituent ingredients of the substantially-anhydrous, dermatologic concentrate of the present invention disclosure, no free water is added to the concentrate. Typically, the water content of the concentrate is less than 1.0% by weight, and is preferably less than 0.5% by weight, and more preferably is less than 0.1%.

The term “dermatologic” is to be understood as suitable for topical application to the skin, hair or scalp of a mammal.

A first essential component of the substantially-anhydrous, dermatologic concentrate of the present invention disclosure are dicarboxylic acids having poor water solubility. These acids are terminally functionalized straight alkyl chains having separation between the carboxylic acid functional groups of from four to ten carbons.

Preferred dicarboxylic acids having poor water solubility suitable for inclusion in the substantially-anhydrous, dermatologic concentrates of the present invention are selected from:
Adipic acid—HOOC(CH₂)₄COOH—four carbons separation.
Azelaic acid—HOOC(CH₂)₇COOH—seven carbons separation.
Sebacic acid—HOOC(CH₂)₈COOH—eight carbons separation.
Dodecanedioic acid—HOOC(CH₂)₁₀COOH—ten carbons separation.

A second essential component of the substantially-anhydrous, dermatologic concentrate of the present invention disclosure are amidoamines, preferably fatty acid amidopropyl dimethylamines manufactured by amidization of fatty acids with 3,3-dimethylaminopropylamine (DMAPA), commonly under alkaline or acidic conditions in the presence of a catalyst. The resultant ingredients have a similar fatty acid amide core structure, with two primary functional groups, a secondary amide and a tertiary amine, separated by a propyl chain.

A first group of preferred fatty acid amidopropyl dimethylamines suitable for inclusion in the substantially-anhydrous, dermatologic concentrates of the present invention are produced by the reaction of DMAPA and fatty acids from: Prunus Dulcis (Almond) Oil; Persea Gratissima (Avocado) Oil; Orbignya oleifera (Babassu) Oil; Brassica Campestris (Rapeseed) Seed Oil; Cocos Nucifera (Coconut) Oil; Avena Sativa (Oat) Kernel Oil; Olea Europaea (Olive) Oil; Sesamum Indicum (Sesame) Oil; Glycine Soja (Soybean) Oil; Helianthus Annuus (Sunflower) Seed Oil; Triticum Vulgare (Wheat) Germ Oil; or Tallow.

A second group of fatty acid amidopropyl dimethylamines suitable for inclusion in the substantially-anhydrous, dermatologic concentrate of the present invention may be produced by the reaction of DMAPA and behenic acid, isostearic acid, lauric acid, linoleic acid, myristic acid, oleic acid, palmitic acid, ricinoleic acid, stearic acid

Another fatty acid amidopropyl dimethylamine suitable for inclusion in the substantially-anhydrous, dermatologic concentrate of the present invention include the condensation product of dilinoleic acid and aminopropyldimethylamine.

Tallamidopropyl dimethylamine, the substituted amine produced by reacting DMAPA and the fatty acids derived from tall oil, may also be used in the substantially-anhydrous, dermatologic concentrate of the present invention

The pH of a topical product made by adding a substantially-anhydrous, dermatologic concentrate in accordance with the first aspect of the present invention disclosure can be alkaline (preferably less than 9); preferably, less than 7; and, more preferably, less than 5.

The pH of a topical product made by adding a substantially-anhydrous, dermatologic concentrate in accordance with the second aspect of the present invention disclosure is less than 6 and is preferably less than 5.

The substantially-anhydrous, dermatologic concentrate of the present invention disclosure can include one or more “functional” or “active” ingredients at concentration(s) that provide(s) one or more skin benefits: agents for the treatment of an inflammatory dermatosis, including acne, psoriasis or rosacea; anti-microbial and anti-fungal actives; anti-itch agents; topical anaesthetics; emollients and skin soothing agents; non-steroidal anti-inflammatory agents; humectants and moisturizing agents, including hyaluronic acid and its derivatives; ingredients that reduce trans-epidermal water loss and/or improve skin barrier function; antioxidants and agents that reduce the appearance of fine lines and wrinkles, including vitamins, proteins and peptides; skin bleaching and lightening agents; and plant-derived ingredients including extracts and oils. Levels of use of these ingredients may be from at least 0.01% on a weight/weight basis based on the weight of the concentrate, at least 0.1%, at least 0.5%, or at least 1.0%.

Preferred, but non limiting examples of “functional” or “active” ingredients include oils, waxes, and esters (including hydrolyzed esters) derived from jojoba seed, and mixtures thereof, commercially available under the tradename LIPONATE® Jojoba NATFILM® from Vantage Specialty Ingredients, Inc. (Vantage Specialty Ingredients, Inc. Warren, NJ (“Vantage”).

Surfactants that can be added to the substantially-anhydrous, dermatologic concentrate of the present invention disclosure may be cationic, anionic, non-ionic, or amphoteric. Non-limiting examples of surfactants include: Sodium Methyl Cocoyl Taurate (METAUPON™ KMT); Magnesium Methyl Cocoyl Taurate; Sodium Methyl Oleoyl Taurate (METAUPON™ OMT); Cocamidopropyl Betaine; Sodium Isethionate; (METAUPON™ SI) Sodium Cocoyl Isethionate; Sodium Lauroyl Sarcosinate; Sodium Cocoyl Glycinate; Disodium Cocoyl Glutamate; Disodium Laureth Sulfosuccinate; Cocamidopropyl Hydroxysultaine. METAUPON™ surfactants are available from Vantage.

In certain preferred embodiments, two or more surfactants (a surfactant blend) are combined with a hydrophilic carrier and the substantially-anhydrous, dermatologic concentrate of the present invention disclosure, for example in the form of an emulsion or a thickened gel (aqueous or hydroglycolic or hydroalcoholic) or a foaming cleanser, bodywash or shampoo. Preferably, the primary surfactant is a taurate, preferably Metaupon® KMT or OMT.

Sodium methyl cocoyl taurate is preferably present on an active matter basis (referred to below “active basis”) at a concentration of from about 6% to about 10%, preferably at least about 7.5%, more preferably at least about 9%.

Sodium methyl oleoyl taurate is preferably present in cleansing compositions at a concentration of from about 1% to about 4% on active basis, preferably at least about 2%.

Non-limiting examples of secondary surfactants include: cocamidopropyl betaine; sodium cocoyl isethionate; sodium lauroyl sarcosinate; sodium cocoyl glycinate; disodium cocoyl glutamate; disodium laureth sulfosuccinate; cocamidopropyl hydroxysultaine.

Certain embodiments contain a film-forming agent, preferably a multifunctional, jojoba-derived sensorial enhancer that provides emolliency, moisturizing, softening and conditioning benefits (among others) and consists essentially of or consists of (i) from about 75 to about 85 parts hydrolyzed jojoba esters (formed by reaction of jojoba oil with an alkali metal hydroxide in water), (ii) from about 8 to about 15 parts jojoba esters, and (iii) from about 7 to about 10 parts water, which is commercially available under the tradename LIPONATE® Jojoba NATFILM® from Vantage.

The multifunctional, jojoba-derived sensorial enhancer is preferably present at a concentration of from about 0.1 wt-% to about 5 wt-%, preferably at a concentration from about 0.5 wt-% to about 3 wt-%, based on the total weight of the finished formulation.

Emulsifiers that can be added to certain embodiments include but are not limited to: oil-in-water emulsifiers (e.g., Ceteareth-20); silicone-in-water emulsifiers (e.g., PEG-12 Dimethicone); water-in-oil emulsifiers (e.g., Polyglyceryl-4 Oleate); water-in-silicone emulsifiers (e.g., PEG/PPG-30/10 Dimethicone or Lauryl PEG/PPG-18/18 Methicone).

As will be appreciated by the person having ordinary skill in the art, viscosity of a finished formulation can be adjusted by the selection of thickening agents, also known in the art as rheological modifiers. Preferred, but not limiting, examples of thickening agents suitable for inclusion in formulations the substantially-anhydrous, dermatologic concentrate of the present invention disclosure include gums, pegylated alkyl glycerides, pegylated esters of fatty acids, fatty alcohols, preferably having fatty chains of 16 or more carbon atoms, and ethoxylated fatty alcohols.

### EXAMPLES

The following examples are illustrative of formulations made with a concentrate of the present invention comprising up to 44% azelaic acid on an active basis. Modifications will be apparent to, and can be readily made by, those skilled in the art without departing from the spirit and scope of the invention. The scope of the appended claims is not to be limited to the examples.

### Example 1—Clear Gel Lotion Containing 1.1% Azelaic Acid

| Phase | INCI | % |
|---|---|---|
| A | Aqua (Water) | 81.45 |
| A | Disodium EDTA | 0.05 |
| B | Jojoba Wax PEG-120 Esters | 3.00 |
| C | Cocamidopropyl Betaine | 8.00 |
| C | Decyl Glucoside | 4.00 |
| C | Concentrate of Present Invention- Cocamidopropyl | 2.50 |
| | Dimethylamine (and) Azelaic Acid (44% active) | |
| C | Caprylyl Glycol (and) Phenoxyethanol (and) | 1.00 |
| | Hexylene Glycol | |
| D | Water (and) Sodium Hydroxide | Q.S. |

### Procedure for Example 1

1. Mix Phase A ingredients in the main vessel with medium speed propeller mixing, no heat.
2. Add Phase B ingredient and mix until fully dispersed.
3. Add Phase C ingredients, one by one. Mix until uniform.
4. Use Phase D ingredient to adjust pH to 5.00.

### Example 2—Emulsion Containing 2% Azelaic Acid

| Phase | INCI | % |
|---|---|---|
| A | Aqua (Water) | 75.80 |
| A | Hydroxyethylcellulose | 0.50 |
| B | Glyceryl Stearate (and) PEG-100 Stearate | 5.00 |
| B | Cetearyl Alcohol | 4.00 |
| B | Cetyl Alcohol | 2.00 |
| B | Caprylic/Capric Triglyceride | 8.00 |
| C | Cocamidopropyl Dimethylamine (and) Azelaic Acid | 4.40 |
| D | Phenoxyethanol (and) Caprylyl Glycol (and) | 0.50 |
| | Hexylene Glycol | |
| E | Water (and) Sodium Hydroxide | Q.S. |

### Procedure for Example 2

1. Premix Phase A ingredients until hydrated.
2. Premix Phase B ingredients.
3. Heat Phases A and B to 70-75 °C.
4. Pour Phase B into Phase A.
5. Add Phase C ingredient at 45 °C.
6. Add Phase D ingredient at 35 °C.
7. Use Phase E ingredient to adjust pH to 4.5-5.5.

### Example 3—Emulsion Containing 14% Azelaic Acid

| Phase | INCI | % |
|---|---|---|
| A | Aqua (Water) | 47.25 |
| B | Xanthan Gum | 1.00 |
| B | Hydroxypropyl Starch Phosphate | 3.50 |
| B | Glycerin | 10.00 |
| C | Caprylic/Capric Triglyceride | 5.00 |
| D | Cocamidopropyl Dimethylamine (and) Azelaic Acid | 32.25 |
| E | Phenoxyethanol (and) Caprylyl Glycol (and) | 1.00 |
| | Hexylene Glycol | |

### Procedure for Example 3

1. Premix Phase B ingredients, then add into Phase A.
2. Heat Phase A (main) to 70-75 °C.
3. Cool down to 45 °C.
4. Add Phase C and Phase D ingredients.
5. Add Phase E ingredient at 35 °C.

### Example 4—Foaming Cleanser Containing 10% Azelaic Acid

| INCI | % |
|---|---|
| Aqua (Water) | 64.1 |
| A. Glycereth-26 | 5.00 |
| B. Sodium Methyl Cocoyl Taurate | 3.00 |
| C. Cocamidopropyl Betaine | 5.00 |
| D. Cocamidopropyl Dimethylamine (and) Azelaic Acid | 22.9 |

Ingredients A-D are miscible in water and in each other. Neither heating nor a specific order of addition (to water) is required to prepare the Cleanser of Example 4.

### Example 5—Emulsified Skin Cream Containing 10% Azelaic Acid

| | |
|---|---|
| 1. Water | 20.55 |
| 2. Concentrate of Present Invention | 22.90 |
| 2. Starch Hydroxypropyltrimonium Chloride | 2.00 |
| 3. Polyether-1 | 0.25 |
| 3. Propanediol | 3.00 |
| 3. Preservative | 1.00 |
| 3. O/W Emulsifier-Malic Acid (and) Polyglyceryl-3 Cetyl Ether | 5.00 |
| (and) Sesamum Indicum (Sesame) Seed Oil | |
| 3. Glycol Distearate | 8.50 |
| 3. Jojoba Esters | 0.20 |
| 3. Jojoba Oil | 0.50 |
| 4. Water | 18.50 |
| 4. Silica | 0.50 |

A sanitized stainless steel jacketed vessel with propeller mixer serves as as a main vessel. Charge the main vessel with water; begin heating to 65 °C. Pre-mix phase 2 into a separate vessel. Add phase 2 pre-mix to phase 1. Pre-mix phase 3 ingredients in a separate vessel. Add phase 3 premix to phase 1-2 mixture at 65 °C. Cool to less than 50 °C; add phase 4. mix to uniformity.

### Example 6—Gel Cleanser

| | | |
|---|---|---|
| 1 | Aqua (Water) | 41.30 |
| 1 | Disodium EDTA | 0.10 |
| 2 | Cocamidopropyl Hydroxysultaine | 2.40 |
| 2 | Water (and) Sodium Methyl Cocoyl Taurate (and) Sodium | 4.00 |
| | Oleoyl Cocoyl Taurate (and) Sodium Lauroyl Sarcosinate* | |
| 2 | Hydrolyzed Jojoba Esters (and) Jojoba Esters | 0.50 |
| | (and) Aqua (Water) | |
| 2 | Concentrate of Present Invention- | 22.80 |
| | Cocamidopropyl Dimethylamine (and) Azelaic Acid* | |
| 3 | Water (and) Sodium Hydroxide | Q.S. |
| 4 | Aqua (Water) (and) Salix Nigra (Willow) Bark Extract | 5.00 |
| 4 | Glycerin (and) Aqua (Water) (and) | 1.00 |
| | Punica Granatum Fruit Extract | |
| 4 | Glycerin (and) Aqua (Water) (and) | 5.00 |
| | Centella Asiatica Extract | |
| 4 | Phenoxyethanol (and) Caprylyl Glycol (and) | 1.00 |
| | Hexylene Glycol | |
| 4 | Parfum (Fragrance) | 0.40 |
| 5 | Xanthan Gum | 4.00 |
| 5 | Glycerin | 8.00 |

Asterisked ingredients available from Vantage under the following tradenames: *-METAUPON™ EZ AMIBIO; **-LIPONATE® Jojoba NATFILM®; and ***-CURAZAELIC™; 44 Vantage Specialty Ingredients, Inc. (Warren, NJ).

Add Phase 1 ingredients to main vessel and mix until uniform. Begin heating to 40 °C. Premix LIPONATE® Jojoba NATFILM® with CURAZELIC™ 44. Add Phase 2 ingredients to main vessel sequentially in order presented, one at a time. Use Phase 3 to adjust pH to 5.5-6.0. Add Phase 4 ingredients to main vessel one at a time. Premix Phase 5 ingredients. Add to Phase 5 pre-mix to main vessel and mix at low-medium speed until fully hydrated. Cool batch.

### Example 7—Acne Spot Treatment

| | | |
|---|---|---|
| 1 | Deionized Water | 66.25 |
| 1 | Cocamidopropyl Dimethylamine | 21.00 |
| | (and) Azelaic Acid | |
| 1 | Cocamidopropyl Dimethylamine (and) | 5.00 |
| | Salicylic Acid | |
| 1 | Hydrolyzed Jojoba Esters (and) Jojoba | 3.00 |
| | Esters (and) (Water) | |
| 1 | Melaleuca Alternifolia (Tea Tree) Leaf Oil | 0.50 |
| 1 | Phenoxyethanol (and) Caprylyl Glycol | 1.00 |
| | (and) Potassium Sorbate (and) | |
| | Hexylene Glycol | |
| 2 | Xanthan Gum | 0.25 |

Asterisked ingredients available from Vantage under the following tradenames: ****-CURCYLIC®

### Example 8—ELISA Immunoassay for Tumor Necrosis Factor-Alpha (TNF-α) and Caspase-1 (CASP-1)

EpiDermFT (EFT-400, MatTek Europe, Bratislava Slovak Republic) is a multilayered reconstituted model of the human dermis and epidermis, formed by culturing normal, human epidermal keratinocytes (NHEK) and normal, human dermal fibroblasts (NHFB). The epidermal compartment of EpiDermFT is analogous to the in vivo cornified epidermal layer with keratin 5 expressing basal cells, and involucrin and keratin 10 expressing spinous and granular layers. The dermal compartment is composed of a collagen matrix containing viable normal human dermal fibroblasts (NHDF). Both the epidermal and dermal layers of EpiDermFT are mitotically and metabolically active and exhibit in vivo-like morphological and growth characteristics.

Testing was conducted on EpiDermFT in 12-well plates filled with culture medium without antibiotics at 37 °C, 5% CO2, saturated humidity (90% RH). Inoculum—6 μL of lipid mix (olive oil and neutral triacylglycerol mixed 1:1) and 60 μL of C. acnes ATCC 11827, 107-108 CFU/mL—was applied to EpiDerm tissue for 48 hours. (Concentration was determined by optical density via spectrophometer at 450 nm.) Excess inoculum was removed and 5 μL of one of three test products was applied to the tissues for a 24-hour treatment period:
Test Group 1: 1% and 2% salicylic acid;
Test Group 2: concentrate of present invention (with azelaic acid as high as 10% on an active basis); and
Test Group 3: 1% and 2% salicylic acid in combination with 5% and 10% azelaic acid

At the end of the treatment period, culture media was collected and stored at −20 °C for subsequent analysis by ELISA immunoassay for two inflammatory markers TNF-a and CASP-1 using commercially-available test kits-Quantikine™ ELISA Human Caspase-1/ICE Immunoassay and Human TNF-α Immunoassay both from R&D Systems, Inc. (Minneapolis, MN), Catalog Number DCA100 and DTAOOD, respectively.

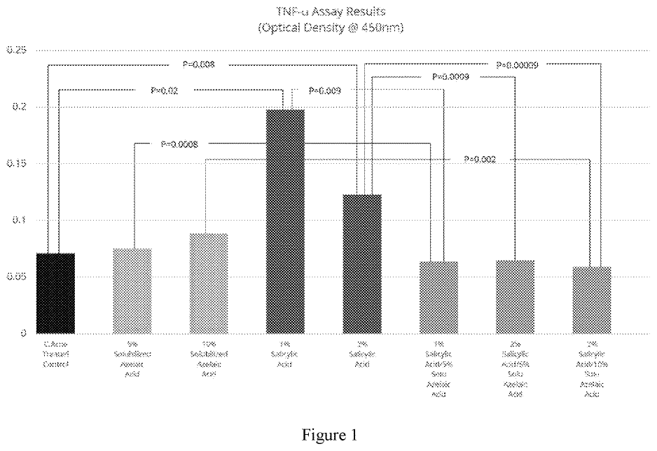

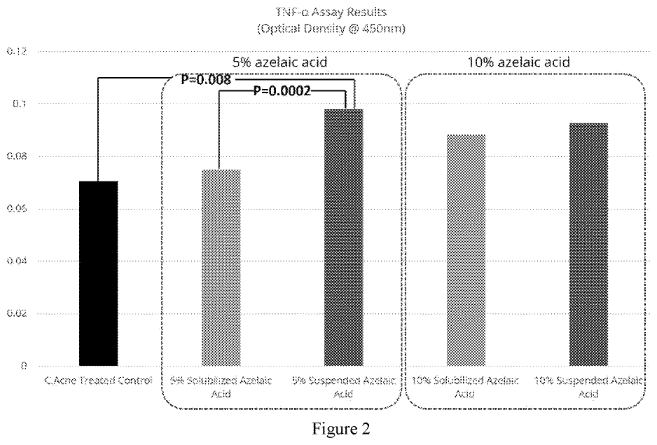

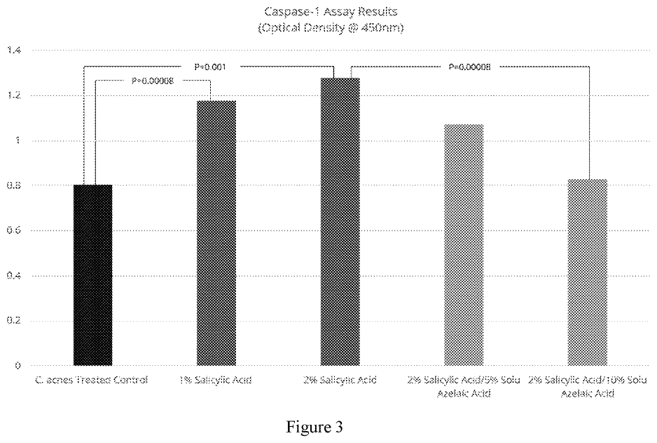

Group 1 exhibited increased inflammatory response versus the C. acnes treated control. Group 2 did not show upregulation of TNF-a versus control. Group 3 showed a measurable decrease in expression of both TNF-a and CASP-1 compared to Group 1. All the results were statistically increased versus the negative control. See FIGS. 1-3.

Concentrate of the present invention was also demonstrated by ELISA to be less inflammatory than prior art suspension having the concentration of azelaic acid on an active basis.

### Example 9—In Vivo Treatment of Post-Inflammatory Hyperpigmentation (PIH)

PIH is a common acquired cutaneous disorder occurring after skin inflammation or injury that is more common and severe in darker-skinned individuals having Fitzpatrick Skin Phototypes (“FST”) III-VI. (The Fitzpatrick Skin Phototype System classifies skin type according to the amount of pigment in skin and the skin's reaction to sun exposure. Fitzpatrick, TB. Soleil et peau. J Med Esthet. 1975; 2:33-34.) PIH results from the overproduction of melanin or an irregular dispersion of pigment after cutaneous inflammation. Grimes PE. Semin Cutan Med Surg. 2009; 28:77-85. In the epidermal compartment, there is an increase in the production and transfer of melanin to surrounding keratinocytes. This increased melanocyte activity has been associated with inflammatory mediators, including TNF-a. Davis EC and VD Callender, J Clin Aesthet Dermatol. 2010; 3 (7): 20.

Sixty subjects (thirty subjects having each of FST V or FST VI) are divided into three groups, each consisting of ten subjects with FST V and ten with FST VI. Two groups are treated with two test products: placebo cream or cream containing concentrate of the present invention for eight weeks. The third untreated group serves as a control. Subjects administer test product (or placebo) once daily for eight weeks. The primary outcome—degree of hyperpigmentation—is objectively measured using the “brown mode” of RBX® Technology on the VISIA® Complexion Analysis System (Canfield Scientific, Parsippany, NJ, USA). RBX® Technology transforms a standard digital image comprised of Red (R), Green (G) and Blue (B) channels into an RBX color-space, where the Red and Brown channels represent hemoglobin and melanin distributions, respectively. Secondary outcomes assessed include: inflammatory and non-inflammatory acne lesion counts evaluated by a dermatologist; skin moisture content (hydration) using a CORNEOMETER® (Courage+Khazaka electronic GmbH (“C+K”), Cologne, Germany); barrier function (trans-epidermal water loss) using a TEWAMETER® (C+K); porphyrin count under UV fluorescence using VISIA®. Measurements are performed at baseline, Week 4, Week 8. Participants also complete a satisfaction question scoring experience and skin tolerability, including burning sensation, itching, and redness (0-10. Results of the study demonstrate that the formulation containing the concentrate of the present invention was able to reduce post-inflammatory hyperpigmentation to a statistically superior level than the placebo formulation or the untreated control group.

1. A substantially-anhydrous, dermatologic concentrate consisting essentially of two constituents:
a. a long-chain alkyl dicarboxylic acid having poor water solubility and having six to twelve carbon atoms selected from the group consisting of adipic acid, azelaic acid, sebacic acid, and dodecanedioc acid; and
b. an amidoamine
wherein the ratio a:b is from 1:9 to 1:1, wherein upon adding the substantially-anhydrous, dermatologic concentrate to water or a hydrophilic carrier a clear solution is formed, having a pH of less than 7, that is visibly free of long-chain alkyl dicarboxylic acid precipitate.

2. The substantially-anhydrous, dermatologic concentrate of claim 1 wherein the ratio of a:b is from 1:4 to 2:3.

3. The substantially-anhydrous, dermatologic concentrate of claim 2 wherein the amidoamine is a fatty acid amidopropyl dimethylamine.

4. The substantially-anhydrous, dermatologic concentrate of claim 3 wherein the fatty acid amidopropyl dimethylamine is cocamidopropyl dimethylamine.

5. The substantially-anhydrous, dermatologic concentrate of claim 1 wherein the long-chain alkyl dicarboxylic acid is azelaic acid.

6. A substantially-anhydrous, dermatologic concentrate consisting essentially of:
a. a long-chain alkyl dicarboxylic acid having poor water solubility and having six to twelve carbon atoms selected from the group consisting of adipic acid, azelaic acid, sebacic acid, and dodecanedioc acid;
b. salicylic acid; and
c. an amidoamine
wherein
i. a and b are present in a combined amount of up to 60 percent by weight of the substantially-anhydrous, dermatologic concentrate;
ii. the ratio of b:c is from 3:7 to 3:2; and
iii. upon adding the substantially-anhydrous, dermatologic concentrate to water or a hydrophilic carrier a clear solution is formed having a pH of less than 6 that is visibly free of (x) long-chain alkyl dicarboxylic acid precipitate and (y) salicylic acid precipitate.

7. The substantially-anhydrous, dermatologic concentrate of claim 6 wherein the amidoamine is a fatty acid amidopropyl dimethylamine.

8. The substantially-anhydrous, dermatologic concentrate of claim 7 wherein the fatty acid amidopropyl dimethylamine is cocamidopropyl dimethylamine.

9. The substantially-anhydrous, dermatologic concentrate of claim 6 wherein the ratio of b:c is from 2:3 to 11:9.

10. The substantially-anhydrous, dermatologic concentrate of claim 9 wherein the alkylamido alkylamine is a fatty acid amidopropyl dimethylamine.

11. The substantially-anhydrous, dermatologic concentrate of claim 10 wherein the fatty acid amidopropyl dimethylamine is cocamidopropyl dimethylamine.

12. The substantially-anhydrous, dermatologic concentrate of claim 6 wherein the long-chain alkyl dicarboxylic acid is azelaic acid.
